(19) United States
(12) Patent Application Publication
(10) Pub. No.: US 2023/0117859 A1
(43) Pub. Date: Apr. 20, 2023

(54) INHALED XENON THERAPY IN NEURODEGENERATIVE DISEASE

(71) Applicants: The Brigham and Women`s Hospital, Inc., Boston, MA (US); General Biophysics LLC, Wayland, MA (US)

(72) Inventors: Ilya Ilin, Wayland, MA (US); Oleg Butovsky, Needham, MA (US); Zhuoran Yin, Boston, MA (US); Howard Weiner, Brookline, MA (US)

(21) Appl. No.: 17/914,061

(22) PCT Filed: Mar. 25, 2021
(86) PCT No.: PCT/US2021/024234

§ 371 Date: Sep. 23, 2022

Related U.S. Application Data
(60) Provisional application No. 63/072,765, filed on Aug. 31, 2020.
(60) Provisional application No. 62/994,548, filed on Mar. 25, 2020.

(51) Int. Cl. A61K 33/00; A61P 25/28; G01N 33/68

(52) U.S. Cl. CPC A61K 33/00; A61P 25/28; G01N 33/6896; G01N 2800/52; G01N 2800/28

(57) ABSTRACT

The present disclosure provides treatments for neurodegenerative disorders and more particularly to methods for treatment of patients with Multiple Sclerosis (MS), Amyotrophic Lateral Sclerosis (ALS), or Alzheimer's disease of different degrees of severity. The methods for treatment of patients who have suffered neurodegenerative diseases and specifically MS, ALS, or Alzheimer's disease includes administering a xenon gas mixture in subjects with elevated levels of neurodegenerative microglia (MGnD), e.g., determined based on levels of inflammatory biomarkers, measured in blood, serum and CSF, or levels of CLEC7A (Dectin-1)/Translocator Protein (TSPO) expression, e.g., measured using TSPO imaging.

### CLAIM OF PRIORITY

This application claims the benefit of U.S. Patent Application Ser. Nos. 62/994,548, filed on Mar. 25, 2020, and 63/072,765, filed on Aug. 31, 2020. The entire contents of the foregoing are hereby incorporated by reference.

### TECHNICAL FIELD

The present disclosure provides medical treatments for the neurodegenerative disorders and more particularly to methods for treatment of patients with Multiple Sclerosis (MS), Amyotrophic Lateral Sclerosis (ALS), or Alzheimer's disease of different degrees of severity. The methods for treatment of patients who have suffered neurodegenerative diseases and specifically MS, ALS, or Alzheimer's disease includes administering a xenon gas mixture in subjects with elevated levels of neurodegenerative microglia (MGnD), e.g., determined based on levels of inflammatory biomarkers, including APOE, SPP1, IGF1, NLRP3, CST3, CST5, CST7, LCN2, CXCL1, CXCL2, CXCL3, CXCL10, CSF1, CSF3, LPL, ITGAX, APP, LYZ2, SERPINB2, MMP3, MMP9, MMP10, MMP13, CH25H, IL1A, IL1B, IL12B, IL6, TNF, EDN1, CD14, CD44, CD300LD, CCL2, CCL3, CCL4, CCL5, CCL6, CCL7, GAS6, LOX e.g., measured in blood, serum and CSF, or levels of CLEC7A (Dectin-1)/Translocator Protein (TSPO) expression, e.g., measured using TSPO imaging.

### BACKGROUND

Alzheimer's disease (AD) is the most prevalent neurodegenerative disorder. Emerging evidence shows that homeostatic dysregulation of the brain immune system, especially that orchestrated by microglia, plays a significant role in the onset and progression of the disease.

Studies showing activated microglia surrounding Aβ plaques in the postmortem AD brain suggest significant involvement of inflammatory pathways in disease progression as well as central role of microglia in Aβ plaques removal. During aging microglia acquire a dystrophic phenotype and/or lose their “sensome” to recognize protein aggregation and fight-off Aβ-plaque accumulation. Amyloid β (Aβ), is considered to play a central role in neuronal cell death. Excess amount of Aβ evokes multiple cytotoxic mechanisms, involving increase of the intracellular Ca2+ level, oxidative stress, and receptor-mediated activation of cell-death cascades. Such diversity in cytotoxic mechanisms induced by Aβ clearly indicates a complex nature of the AD-related neuronal cell death. Current approaches are entirely targeting intracellular neurofibrillary tangles and extracellular plaques composed primarily of Aβ. However, in addition to removal of the toxic Aβ, direct suppression of neuronal loss is needed for AD treatment and no such neuroprotective therapies have been developed. Preservation of neuronal cells from Aβ induced apoptosis as well as restoration of resident microglial homeostatic function is critical for the restoration of brain function.

Amyotrophic lateral sclerosis (ALS) is another neurodegenerative disease characterized by deep involvement of cell mediating neuroinflammatory processes. The treatments with the aim of reducing the pro-inflammatory action of microglia and astrocytes were tested in the animal models of ALS and have been modestly successful. Similar to AD during ALS, microglia display different phenotypes at the different stages of the disease.

Multiple sclerosis (MS) is a complex inflammatory disease accompanied by demyelination of the central nervous system. It has been acknowledged that MS is a combination of inflammatory processes and neurodegeneration, typically at later stages of the disease. MS was once traditionally thought of a predominantly T-cell-mediated autoimmune disease. It is now known that microglia, the resident CNS immune cells, are key players in MS disease progression.

### SUMMARY

The present disclosure provides methods for patients who suffer from neurodegenerative diseases of different degrees of severity. Without wishing to be bound by theory, the present methods prevent inflammation by preserving microglia in the homeostatic form and simultaneous protection of neuronal cells from apoptosis due to the employment of the protective action of xenon. The present methods are directed at restoration of microglial homeostatic function, preventing accompanying inflammation.

Thus, provided herein are methods for treating a subject that can include identifying a subject who has a level of microglial cells that express C-type lectin domain family 7 member A (CLEC7a) above a reference level; and administering a therapeutically effective amount of xenon to the subject, e.g., using repeated administration of xenon by inhalation, e.g., wherein the xenon is administered to the subject in a gas for inhalation. Also provided herein is the use of xenon in a method described herein.

In some embodiments, the subject has, or is at risk of developing, a neurodegenerative disease. In some embodiments, the neurodegenerative disease is Alzheimer's disease, Multiple Sclerosis (MS), or Amyotrophic Lateral Sclerosis (ALS).

In some embodiments, identifying a subject who has levels of Clec7a+ microglial cells above a reference level comprises measuring expression of translocator protein 18 kDa (TSPO) in a tissue of the subject, e.g., the brain of the subject to determine a level of Clec7a+ microglial cells in the tissue; comparing the level of TSPO expression in the tissue to a reference level; and identifying a subject who has a level of TSPO expression above the level as having a level of Clec7a+ microglial cells above the reference level.

In some embodiments, identifying a subject who has levels of Clec7a+ microglial cells above a reference level comprises: measuring levels of one or more inflammatory biomarkers selected from apolipoprotein E (APOE); secreted phosphoprotein 1 (SPP1); insulin like growth factor 1 (IGF1); NLR family pyrin domain containing 3 (NLRP3); cystatin C (CST3); cystatin D (CST5); cystatin F (CST7); lipocalin 2 (LCN2); C-X-C motif chemokine ligand 1 (CXCL1); C-X-C motif chemokine ligand 2 (CXCL2); C-X-C motif chemokine ligand 3 (CXCL3); C-X-C motif chemokine ligand 10 (CXCL10); colony stimulating factor 1 (CSF1); colony stimulating factor 3 (CSF3); lipoprotein lipase (LPL); integrin subunit alpha X (ITGAX); amyloid beta precursor protein (APP); lysozyme 2 (LYZ2); serpin family B member 2 (SERPINB2); matrix metallopeptidase 3 (MMP3); matrix metallopeptidase 9 (MMP9); matrix metallopeptidase 10 (MMP10); matrix metallopeptidase 13 (MMP13); cholesterol 25-hydroxylase (CH25H); interleukin 1 alpha (IL1A); interleukin 1 beta (IL1B); interleukin 12B (IL12B); interleukin 6 (IL6); tumor necrosis factor (TNF); endothelin 1 (EDN1); CD14; CD44; CD300 molecule like family member d (CD300LD); C-C motif chemokine ligand 2 (CCL2); C-C motif chemokine ligand 3 (CCL3); C-C motif chemokine ligand 4 (CCL4); C-C motif chemokine ligand 5 (CCL5); C-C motif chemokine ligand 6 (CCL6); C-C motif chemokine ligand 7 (CCL7); growth arrest specific 6 (GAS6); lysyl oxidase (LOX) in a sample from the subject, preferably a sample comprising blood from the subject to determine; comparing the level of the inflammatory biomarker in the sample to a corresponding reference level; and identifying a subject who has a level of APOE, SPP1, IGF1, NLRP3, CST3, CST5, CST7, LCN2, CXCL1, CXCL2, CXCL3, CXCL10, CSF1, CSF3, LPL, ITGAX, APP, LYZ2, SERPINB2, MMP3, MMP9, MMP10, MMP13, CH25H, IL1A, IL1B, IL12B, IL6, TNF, EDN1, CD14, CD44, CD300LD, CCL2, CCL3, CCL4, CCL5, CCL6, CCL7, or LOX that is above the reference level, or a level of GAS6 that is below the reference level as having a level of Clec7a+ microglial cells above the reference level.

In some embodiments, the methods include determining a subsequent level of Clec7a+ microglial cells after administration of the xenon, and administering a further dose of xenon if the subsequent level of Clec7a+ microglial cells is above a reference level.

In some embodiments, the xenon is administered to the subject in a gas for inhalation. In some embodiments, the gas comprises at least 25%, 30%, 35%, 40%, 45%, 50%, 55%, 60%, 65%, up to 70% xenon, and at least 20%, 21%, 25%, 30%, 40% oxygen.

In some embodiments, the xenon is administered for at least 30 minutes, 45 minutes, one hour, or two hours. In some embodiments, the xenon is administered daily, once a week, twice a week, every other week, once a month, or once every two months. In some embodiments, the xenon is administered once, twice, three times, or four times a week or more. In some embodiments, the administration is repeated for at least two, three, four, five, 6, 7, 8, 9, 10, 11, or 12 weeks. In some embodiments, the administration is repeated every other week for at least eight weeks (i.e., four weeks on, four weeks off). In some embodiments, the treatment reduces levels of Clec7a+ microglial cells in the subject. In some embodiments, the treatment reduces inflammation, e.g., neuroinflammation in the subject.

Thus provided herein are methods for the treatment of patients who have suffered neurodegenerative diseases of different degrees of severity by modulating microglial cells comprising the steps of: a) identifying a patient with elevated levels of MGnD (e.g., Clec7a+) microglial cells, b) administering gas composition containing xenon gas periodically for the time period of one hour, c) measuring Clec7a+ microglial cell count periodically and adjusting frequency of gas administration.

Also provided are methods for treatment of patients to enhancing the survival ability of neuron cells due to simultaneous reduction of their apoptosis and modulating microglial cells due to the employment of the protective action of xenon consisting of steps of: a) identifying a patient with elevated levels of MGnD (e.g., Clec7a+) microglial cells b) administering gas composition containing xenon gas periodically for the time period of one hour, c) measuring Clec7a+ microglial cell count periodically and adjusting frequency of gas administration. In some embodiments, the neurodegenerative disease is Alzheimer's disease of different degrees of severity. In some embodiments, the neurodegenerative disease is MS or ALS of different degrees of severity.

In some embodiments, the level of Clec7a+ microglial cells is determined by measuring Translocator Protein (TSPO), or by identifying a patient by determining levels of one, two, three, four, five, six or more of APOE¹, SPP1, IGF1, NLRP3, CST3, CST5, CST7, LCN2, CXCL1, CXCL2, CXCL3, CXCL10, CSF1, CSF3, LPL, ITGAX, APP, LYZ2, SERPINB2, MMP3, MMP9, MMP10, MMP13, CH25H, IL1A, IL1B, IL12B, IL6, TNF, EDN1, CD14, CD44, CD300LD, CCL2, CCL3, CCL4, CCL5, CCL6, CCL7, GAS6, LOX measured in blood, serum or cerebrospinal fluid, and identifying a subject who has a level of APOE, SPP1, IGF1, NLRP3, CST3, CST5, CST7, LCN2, CXCL1, CXCL2, CXCL3, CXCL10, CSF1, CSF3, LPL, ITGAX, APP, LYZ2, SERPINB2, MMP3, MMP9, MMP10, MMP13, CH25H, IL1A, IL1B, IL12B, IL6, TNF, EDN1, CD14, CD44, CD300LD, CCL2, CCL3, CCL4, CCL5, CCL6, CCL7, LOX that is above a reference level, or a level of GAS6 that is below a reference levels, and selecting that subject for treatment.

In some embodiments, a gas mixture containing xenon gas is delivered via inhalation by the patient.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Xe-gas treatment to restore M0-homeostatic functional microglia to treat AD. APOE signaling induces microglia phenotype switch from M0-homeostatic to MGnD-neurodegenerative via suppression of Mef2a and activation of Bhlhe40 and miR-155 associated with dystrophic axons or Ab dimers in AD. Xe-treatment restores functional M0-microglia in APP-PS1 mice.

FIGS. 2A-D: APOE is highly expressed in Clec7a^{+ MGnD microglia associated with Aβ-plaques. (A) Immunohistochemistry of Clec}7a and P2ry12 in APP-PS1 mice at 4 months of age. Arrows show transition from M0 (P2ry12⁺) to MGnD (Clec7a^{+) MGnD phenotype. (B) Isolation of Clec}7a⁺/FCRLS⁺ and Clec7a⁻/FCRLS⁺ microglia from APP-PS1 mice. (C) Heatmap of identified DEGs in A□-plaque associated (Clec7a⁺/FCRLS⁺) vs non-plaque (Clec7a⁻/FCRLS⁺) microglia by RNA-seq analysis. (D) Staining for P2ry12 with Aβ or pNF in diffuse versus neuritic plaques in human AD brain.

FIGS. 3A-D. Xenon treatment of APP-PS1 mice. A. Xe-treatment chamber. Closed circuit system with pump recirculated gas. The gas mixture was premixed 70% Xe and 20% O2. B. Experimental design. C. Heatmap of significantly changed genes (p<0.05) in dN-phagocytic microglia exposed to Xenon vs. normal air in vivo as determined by RNA-Sequencing. Blue: down-regulated genes; Red: up-regulated genes. Significance determined by Student t-test. D. Xe treatment suppression of the specific genes.

FIGS. 4A-E. Xenon treatment reduced Clec7a^{+ MGnD-neurodegenerative microglia subset and Ab load in APP/PS}1 mice. A. Representative images from the cortex of the APP/PS1 mice exposed to Xenon vs. normal air stained with 6E10 (red). Bar=100 mm. B. Quantification of the area of 6E10⁺ Ab plaques in the cortex area. C. Quantification of number of the size of 6E10⁺ plaques according to different plaque sizes in the cortex area. D. Representative images from the cortex APP/PS1 mice exposed to Xenon vs. normal air stained with Clec7a (green). Bar=100 mm. E. Quantification of the area of Clec7a⁺ staining in the cortex. Data are presented as mean±SEM. Student's t-test was used for statistics. * p<0.05.

FIGS. 5A-B. Induction of Clec7a and TSPO on MGnD microglia associated with neuritic Ab-plaques. Scatter plots with bar showed mRNA transcripts of Tspo (5A) and Clec7a (5B) in Clec7a⁺ (plaque-associated) versus Clec7a⁻FCRLS⁺ (non-plaque associated) microglia in APP-PS1 mice versus FCRLS⁺ microglia in WT mice (N=6, 24 months old, mean±SEM). ****p<0.0001 by one-way ANOVA followed by Tukey's multiple-comparison post-hoc test.

FIGS. 6A-B. Xenon treatment decreased Apoe phagocytosed by CD68⁺ phagosomes in APP/PS1 mice. A. Representative confocal images from the cortex APP/PS1 mice exposed to Xenon vs. normal air in vivo stained with Apoe (red) and CD68 (gray), displaying CD68⁺ cells phagocytosing Apoe around plaques. B. Percentage of Apoe engulfed in CD68⁺ phagosomes in both groups. 5-9 plaques per sample were analyzed. Data are presented as mean±SEM. Student's t-test was used for statistics. * p<0.05.

FIGS. 7A-D. Xe-treatment prevents apoptosis in variety of cells. A. Viability of lymphocyte-like Jurkat cell after 1 day storage. B. Xenon inhibited apoptosis in stored U-937 cells (NG—no gas means no xenon). C. Viability of monocyte-like U-937 cells after 1 day storage. D. Viability of monocyte-like U-937 cells after 14 days storage.

FIG. 8. Xenon treatment induces homeostatic and phagocytosis-related genes in MG-dNΦ. Heatmap of significantly changed genes (p<0.05) in dN-phagocytic microglia exposed to Xenon vs. normal air in vivo as determined by RNA-Sequencing. N=3 (Xenon), 4 (Air), Significance determined by Student t-test.

FIGS. 9A-E. Xenon treatments delayed EAE symptoms. A. Scheme representative of experimental design used. C57BL6 animals were immunized with 150 ug of MOG diluted v/v in CFA followed by two doses of B. Pertussis toxin (200 ng) applied during immunization and 48 hours later. The treatment with xenon consisted of a 40 minutes exposure twice a week starting on day 0. After clinical evaluation the animals were euthanized at the peak of the disease and the frequency of inflammatory cells evaluated by flow cytometry and IHC. B. Score curve from clinical signs. C. Evaluation of microglia cells from spinal cord by flow cytometry. Gate based on population CD45+, CD11b+, Ly6C−, Clec12a−. D. T Helper cells evaluation by flow cytometry. E. Immunofluorescence of spinal cord from animals on day 17^{th}. At top the control animals and at the bottom the animals treated with Xenon. The tissue was stained for Tmem119, Clec7a and MBP, quantification measured by the Fiji ImageJ software. Statistical analysis: T-test.

FIGS. 10A-B. Xenon treatment suppresses cytokines in MGnD-neurodegenerative microglia associated with neurotic Aβ-plaques in APP/PS1 mice. A) List of cytokines induced in Aβ-plaque-associated microglia in APP/PS1 mice; B) Xenon treatment suppresses cytokines induced in MGnD-microglia associated with neurotic Aβ-plaques in APP/PS1 mice, such as Spp1, Apoe, and Igf1.

### DETAILED DESCRIPTION

Emerging evidence shows that homeostatic dysregulation of the brain's immune system, especially that orchestrated by microglia, plays a significant role in the onset and progression of neurodegenerative diseases including AD, MS, and ALS. For example, microglial function that is maintained in healthy brains is pathogenically dysregulated in AD brain. The prominent genetic risk factor, APOE, is involved in microglial function. A microglial phenotype switch from homeostatic (M0) to neurodegenerative (MGnD)²⁻⁵ is mediated by APOE-signaling⁶. This phenotype was associated with reciprocal activation of the APOE and suppression of TGFβ signaling—a key regulator of M0 microglia³⁻⁷. Human amyloid-b (Ab)-dimers isolated from AD brains or phagocytosis of apoptotic neurons suppress the homeostatic molecules and activates inflammatory molecules including APOE as the most upregulated molecule in microglia. Transcription regulatory network analysis showed that APOE and miR-155 suppress PU.1, MEF2A, SMAD3 and TGFβ signaling which are the core transcription regulators in M0-microglia^{3,7,8} and induces BHLHE40, which is essential for pathogenicity in neuroinflammation^{9,10}. Genetic targeting of Apoe restores TGFβ-dependent M0-homeostatic microglia. Moreover, conditional deletion of Apoe specifically in phagocytic microglia showed a similar pattern of expression of selected genes as was observed in phagocytic microglia from global Apoe^{-/-} mice. Global genetic ablation of mouse Apoe and especially human APOE4 in P301S tau transgenic mice restored homeostatic microglia and arrested neurodegeneration and brain atrophy¹¹.

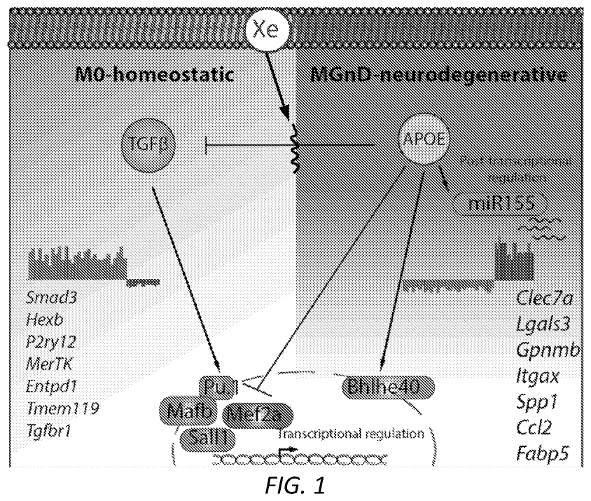

Recent studies showed that Xenon (Xe) gas treatment has a neuroprotective role^{12-15,37}. Xe is currently used in human patients as an anesthetic and as a neuroprotectant in treatment of brain injuries. Xe penetrates the blood brain barrier, making it a potentially effective therapeutic. As shown herein, Xe delivered through inhalation modulates microglial phenotype, pushing the balance towards repair (FIG. 1).

Methods of Treatment

Provided herein are methods for treating subjects with a neurodegenerative disease using xenon, e.g., inhaled xenon gas. In some embodiments, the neurodegenerative disease is Alzheimer's disease, amyotrophic lateral sclerosis (ALS), or multiple sclerosis (MS). In some embodiments, the disease is advanced, e.g., advanced AD, ALS, or MS. In some embodiments, the methods halt, slow, or reduce the risk of progression; in some embodiments, the methods result in an improvement in one or more clinical parameters or symptoms of the disease, e.g., in Cognitive ability in AD and motor function in MS and ALS. The methods can include identifying a subject for treatment using Xenon gas as described herein, by detecting elevated levels of MGnD. The methods can include detection of levels of Clec7 (e.g., by detecting increased levels of TSPO) or other biomarkers of MGnD as described herein in the subject.

The methods include administering a therapeutically effective amount of xenon, e.g., via inhalation. The methods can include administering a gas that comprises at least 25%, 30%, 35%, 40%, 45%, 50%, 55%, 60%, 65%, 70%, 75%, or 80% xenon, and at least 16%, 20%, 21%, 25%, 30%, 40% oxygen. The balance of the gas can be, e.g., nitrogen, carbon dioxide, neon, and hydrogen, or other gases that make up the air of the room in which the subject is being treated.

The xenon can be administered in doses of at least 30 minutes, 45 minutes, one hour, or two hours or longer.

The xenon can be administered once or twice a day, e.g., daily, once a week, twice a week, every other week, once a month, or once every two months.

In some embodiments, in each week the xenon is administered once, twice, three times, or four times a week or more. In some embodiments, the administration is repeated, e.g., for at least two, three, four, five, 6, 7, 8, 9, 10, 11, 12, 18, or 24 weeks, or longer, e.g., for years, e.g., for the lifetime of the subject. In some embodiments, the xenon treatment or treatments are administered on alternating weeks, or alternating bi-weeks, where two weeks of treatment are followed by two weeks without treatment. In some embodiments, the administration is repeated every other week for at least eight weeks (i.e., four weeks on, four weeks off).

In some embodiments, the xenon introduction to the brain is via inhalation by the patient. In some embodiments, the xenon introduction via inhalation by the patient is by a gas mixture containing two or more gasses selected from the group consisting of xenon, oxygen and nitrogen. The content of the xenon in the gas mixture is at least about 5 vol. %, generally less than about 70 vol. %, typically about 2-60 vol. %, and more typically about 5-50 vol. %. The content of oxygen, when used is at least about 1 vol. %, generally less than about 40 vol. %, typically about 5-30 vol. %, and more typically about 18-25 vol. % (e.g., 21 vol. %). The content of nitrogen, when used, is at least about 10 vol. %, generally less than about 99 vol. %, typically about 20-85 vol. %, and more typically about 29-75 vol. %. In one non-limiting formulation, the gas mixture includes xenon, oxygen and nitrogen. In another and/or alternative non-limiting formulation, 95-100 vol. % of the gas mixture includes xenon, oxygen and nitrogen. In still another and/or alternative non-limiting formulation, nitrogen, when used, constitutes the largest volume percent of the gas mixture. In yet another and/or alternative non-limiting formulation, nitrogen, when used, constitutes over 50 vol. % of the gas mixture. In still yet another and/or alternative non-limiting formulation, the volume percent of oxygen, when used, is greater than the volume percent of the xenon in the gas mixture. In another and/or alternative non-limiting formulation, the volume percent of nitrogen, when used, is greater than the volume percent of the oxygen, when used, in the gas mixture. In still another and/or alternative non-limiting formulation, the volume percent of nitrogen, when used, is greater than the combined volume percent of oxygen, when used, and xenon in the gas mixture.

In some embodiments, the xenon introduction via inhalation by the patient is introduced to the patient at a pressure that is at atmospheric pressure (e.g., 600-760 mmHg depending on ambient elevation) or at a pressure exceeding atmospheric pressure (e.g., atmospheric pressure plus 600-1550 mmHg, etc.). In some embodiments, the xenon introduction via inhalations by the patient is introduced to the patient at atmospheric pressure plus 0-1400 mmHg. In some embodiments, the xenon introduction via inhalations by the patient is introduced to the patient at atmospheric plus 1-1400 mmHg.

In some embodiments, the xenon introduction via inhalations by the patient is introduced to the patient for a time period of at least about 10 minutes. In some embodiments, the xenon introduction via inhalations by the patient is introduced to the patient for a time period of about 10-300 minutes. In some embodiments, the xenon introduction via inhalations by the patient is introduced to the patient for a time period of about 30-250 minutes. In some embodiments, the xenon introduction via inhalations by the patient is introduced to the patient for a time period of about 60-180 minutes.

In some embodiments, the xenon introduction via inhalations by the patient is introduced to the patient repeatedly once a day. In some embodiments, the xenon introduction via inhalations by the patient is introduced to the patient repeatedly twice a day. In some embodiments, the xenon introduction via inhalations by the patient is introduced to the patient repeatedly three times per day. In some embodiments, the xenon introduction via inhalations by the patient is introduced to the patient repeatedly two times per week. In some embodiments, the xenon introduction via inhalations by the patient is repeatedly introduced to the patient as described above for a time period of about a month. In some embodiments, the xenon introduction via inhalations by the patient is repeatedly introduced to the patient as described above for a time period of about three months. In some embodiments, the xenon introduction via inhalations by the patient is repeatedly introduced to the patient as described above indefinitely or for a period of time of at least one year. In some embodiments, the xenon introduction via inhalations by the patient is continuously introduced to the patient, e.g. together with respiration gas mixture, containing oxygen or separately as an independent gas mixture.

In some embodiments, the xenon introduction via inhalations by the patient can be introduced to the patient at a flowrate of xenon of at least about 0.1 liter per minute. In some embodiments, the flowrate of xenon is up to about 200 l/min. In some embodiments, the flowrate of xenon is about 1-120 l/min. (See M. N. Zamyatin. Using Xenon Anesthesia in a Multidisciplinary Hospital—Proceedings of the Second Conference of Anesthesiologists and Intensivists of medical institutions of the Russian Ministry of Defense. M.: Military Clinical Hospital named after N. N. Burdenko. 2010, pp. 83-100; U.S. Pat. No. 6,536,429). Using higher concentration of xenon may lead to narcotic sleep or temporary impairment of consciousness and, as with any anesthetic, can lead to variation of such indicators of cardiovascular system as arterial pressure and heart rate. The use of gas mixtures characterized by low concentrations of xenon do not lead to changes in patient's consciousness, do not have anesthetic or narcotic action and do not require permanent monitoring by anesthesiologist.

In some embodiments, the xenon introduction via inhalations by the patient can be by face mask, nasal cannula and the like. Face mask inhalation can be conducted, similar to the providing patient with the oxygen, through the mask connected directly with a regulator of the gas tank or with the use of anesthesia machines. Generally, the pressure of the gas supplied by such machines can be up to 2 atmospheres. The advantage of this inhalation option is an ability to collect exhaled gas mixture. Xenon gas in exhaled mixture then can be collected and recirculated reducing its consumption for each procedure. One disadvantage of this approach is relative inconvenience. Nasal cannula inhalation (which can be conducted with pressure values being within the same range as face mask anesthesia) represents another delivery option. However, it can be difficult to collect exhaled gas and recirculate xenon gas. The advantages of this option are the convenience and ability to provide longer treatment or continuous gas support to the patient without significant disruption of patient's daily activity. One non-limiting example of the application of the methods described herein is set forth as follows:

Without wishing to be bound by theory, the methods described herein are believed to preserve neuronal cells from Aβ induced apoptosis as well as restore resident microglial homeostatic function, and in some embodiments can include the use of the following protocol for xenon treatment:

In some embodiments, in the course of treatment, the patient practices inhalations with gas mixture two times per day. The gas mixture contains 21-23 vol % oxygen, 40-70 vol % of xenon, and 7-29 vol % of nitrogen for the balance of the gas mixture. Every inhalation is delivered through the inhalation face mask. The gas mixture is fed to the patient for a period of 30 minutes to 1 hour at a pressure of atmospheric pressure plus 0-760 mmHg. Such daily treatment is continued for the period of at least three months. It was established that xenon which is fed to the patient via inhalations can be successfully used to restore resident microglial homeostatic function and therefore reduce inflammation which increases neuronal damage. It also protects neuronal cells from apoptosis.

In some embodiments, the patient inhales xenon gas mixture continuously through the nasal cannula. The gas mixture contains 21-44 vol % of oxygen, 5-50 vol % of xenon and the rest of nitrogen. In still another and/or alternative non-limiting arrangement, the patient inhales xenon gas mixture through the nasal cannula, where xenon gas is premixed with ambient air in concentration of 5-50 vol % of xenon and the rest 95-50 vol % of air.

In some embodiments, the xenon gas, exhaled by the patient in the course of the treatment, is captured and recycled.

Described herein are methods for treatment of patients who have suffered neurodegenerative diseases of different degrees of severity, including methods for treatment of patients who suffer from Alzheimer's disease, MS, or ALS of different degrees of severity.

Also provided herein are methods for treatment of patients to reduce inflammation and enhance the survival ability of neuron cells by modulating microglial cells due to employment of the protective action of xenon.

In some embodiments provided herein are methods for treatment of patients to enhance the survival ability of neuron cells due to reduction of their apoptosis and modulating microglial cells due to the employment of the protective action of xenon.

Also provided herein are methods for treatment of patients that involve periodic inhalation by the patient of a gas mixture containing xenon gas, or continuous inhalation by the patient of a gas mixture containing xenon gas.

Further, provided are methods for treatment of patients that involve a combination of periodic inhalation by the patient of gas mixture containing xenon gas and continuous inhalation by the patient of gas mixture containing xenon gas.

Therefore, these data demonstrate that xenon gas treatment modulates microglial phenotype which pushes the balance towards repair. Thus, xenon gas treatment has a protective immunomodulatory role to induce microglia protective functions to treat AD.

The above-given experimental data verifies that the inhalation with xenon containing gas mixture for a limited period of time (e.g. 1 hour) protects homeostatic form of microglia and induces processes associated with restriction of AD.

Inhalation (conducted using one of the above-described approaches: face mask or nasal cannula) can be arranged. For example, face mask inhalation which uses a gas mixture of oxygen—approximately 21%, xenon—approximately 5-70%, and the balance nitrogen can be used. The gas mixture can be supplied to the face mask under pressure exceeding the atmospheric pressure by no more than about 2 atmospheres (excess pressure) and with the xenon flow rate being no less than approximately 0.1 l/min and generally about 1 l/min to 120 l/min depending on the patient's health state.

The inhalation generally is conducted every day, once, twice or three times a day. The duration of inhalation generally is about 1-3 hours. Multiple inhalations can be used. For example, the next of a subsequent inhalation could be conducted within 0.01-4 hours from the termination of the first or previous inhalation. The number of inhalation sections (e.g., 1, 2, 3, etc.) is not limiting, thus should be specified by a physician based on the patient's health condition. The total treatment period with daily inhalations can be 1 month, 2 months, 3 months, and longer depending on the patient's health condition determined by the physician.

In some embodiments, the nasal cannula is used instead of face mask inhalation; however, this is not required. The course duration is not limited and is determined by patient's health condition. Nasal cannula inhalation allows using this method for patients continuously without significant disturbance of their daily life routine.

Methods known in the art can be used to deliver the xe gas; in some embodiments, an inhalation device is used.

Biomarkers of MGnD

Included herein are methods for identifying or selecting subjects for, or predicting response to, treatment using a method described herein. The methods can include detection of levels of MGnD (i.e., Clec7A+ MGnD) in the subject.

The methods can include obtaining a sample comprising microglia from a subject, and evaluating the presence and/or level of Clec7 in the sample, and comparing the presence and/or level with one or more references, e.g., a control reference that represents a normal level of Clec7, e.g., a level in an unaffected subject, and/or a disease reference that represents a level of Clec7 associated with the presence of MGnD, e.g., a level in a subject having AD, who is likely to respond to treatment using a method described herein. As noted below, TSPO measurements can be used to determine levels of Clec7A+ MGnD.

The methods can also or alternatively include determining a level of other inflammatory biomarkers correlated with levels of Clec7A+ MGnD. Such methods can include obtaining a sample of blood, serum or cerebrospinal fluid (CSF) and measuring levels of one, two, three, four, five, six or more of APOE¹, SPP1¹⁶, IGF1, NLRP3¹⁷, CST3, CST5, CST7, LCN2¹⁸, CXCL1, CXCL2¹⁹, CXCL3, CXCL10²⁰, CSF1, CSF3, LPL²¹, ITGAX, APP, LYZ2, SERPINB2, MMP3, MMP9^{22,23}, MMP10, MMP13, CH25H, IL1A, IL1B, IL12B, IL6, TNF²⁴, EDN1, CD14, CD44, CD300LD, CCL2²³, CCL3, CCL4, CCL5, CCL6, CCL7, GAS6²⁵, LOX, and identifying a subject who has a level of APOE, SPP1, IGF1, NLRP3, CST3, CST5, CST7, LCN2, CXCL1, CXCL2, CXCL3, CXCL10, CSF1, CSF3, LPL, ITGAX, APP, LYZ2, SERPINB2, MMP3, MMP9, MMP10, MMP13, CH25H, IL1A, IL1B, IL12B, IL6, TNF, EDN1, CD14, CD44, CD300LD, CCL2, CCL3, CCL4, CCL5, CCL6, CCL7, LOX that is above a reference level, or a level of GAS6 that is below a reference levels, and selecting that subject for treatment.

The following table provides exemplary human sequences for each of the biomarkers.

| | GenBank RefSeq ID | | |
|---|---|---|---|
| Gene | Nucleic Acid | Protein | Notes |
| TSPO | NM_000714.6 | NP_000705.2 | This variant (PBR) |
| | | | consists of four exons |
| | | | and encodes the protein |
| | | | PBR. |
| CLEC7A | NM_197947.3 | NP_922938.1 | C-type lectin domain |
| | | | family 7 member A |
| | | | isoform a; This variant |
| | | | (1) represents the |
| | | | longest transcript and |
| | | | encodes the longest |
| | | | isoform (a). |
| APOE | NM_001302688.2 | NP_001289617.1 | apolipoprotein E |
| | NM_000041.4 | NP_000032.1 | |
| | NM_001302689.2 | NP_001289618.1 | |
| | NM_001302690.2 | NP_001289619.1 | |
| | NM_001302691.2 | NP_001289620.1 | |
| SPP1 | NM_001040058.2 | NP_001035147.1 | secreted |
| | | | phosphoprotein 1 |
| IGF1 | NM_000618.5 | NP_000609.1 | insulin like growth |
| | | | factor 1 |
| NLRP3 | NM_001243133.2 | NP_001230062.1 | NLR family pyrin |
| | | | domain containing 3 |
| CST3 | NM_000099.4 | NP_000090.1 | cystatin C |
| CST5 | NM_001900 | NP_001891 | cystatin D |
| CST7 | NM_003650 | NP_003641 | cystatin F |
| LCN2 | NM_005564.5 | NP_005555.2 | lipocalin 2 |
| CXCL1 | NM_001511.4 | NP_001502.1 | C-X-C motif |
| | | | chemokine ligand 1 |
| CXCL2 | NM_002089.4 | NP_002080.1 | C-X-C motif |
| | | | chemokine ligand 2 |
| CXCL3 | NM_002090.3 | NP_002081.2 | C-X-C motif |
| | | | chemokine ligand 3 |
| CXCL10 | NM_001565.4 | NP_001556.2 | C-X-C motif |
| | | | chemokine ligand 10 |
| CSF1 | NM_000757.6 | NP_000748.4 | colony stimulating |
| | | | factor 1 |
| CSF3 | NM_172219.3 | NP_757373.1 | colony stimulating |
| | | | factor 3 |
| LPL | NM_000237.3 | NP_000228.1 | lipoprotein lipase |
| ITGAX | NM_000887.5 | NP_000878.2 | integrin subunit alpha X |
| APP | NM_000484.4 | NP_000475.1 | amyloid beta precursor |
| | | | protein |
| LYZ2 | NM_017372.3 | NP_059068.1 | lysozyme 2 |
| SERPINB2 | NM_002575.3 | NP_002566.1 | serpin family B member 2 |
| MMP3 | NM_002422.5 | NM_002422.5 | matrix metallopeptidase 3 |
| MMP9 | NM_004994.3 | NP_004985.2 | matrix metallopeptidase 9 |
| MMP10 | NM_002425.3 | NP_002416.1 | matrix metallopeptidase 10 |
| MMP13 | NM_002427.4 | NP_002418.1 | matrix metallopeptidase 13 |
| CH25H | NM_002425.3 | NP_002416.1 | cholesterol 25-hydroxylase |
| IL1A | NM_000575.5 | NP_000566.3 | interleukin 1 alpha |
| IL1B | NM_000576.3 | NP_000567.1 | interleukin 1 beta |
| IL12B | NM_002187.3 | NP_002178.2 | interleukin 12B |
| IL6 | NM_000600.5 | NP_000591.1 | interleukin 6 |
| TNF | NM_000594.4 | NP_000585.2 | tumor necrosis factor |
| EDN1 | NM_001955.5 | NP_001946.3 | endothelin 1 |
| CD14 | NM_000591.4 | NP_000582.1 | |
| CD44 | NM_000610.4 | NP_000601.3 | |
| CD300LD | NM_001115152.2 | NP_001108624.1 | CD300 molecule like |
| | | | family member d |
| CCL2 | NM_002982.4 | NP_002973.1 | C-C motif chemokine |
| | | | ligand 2 |
| CCL3 | NM_002983.3 | NP_002974.1 | C-C motif chemokine |
| | | | ligand 3 |
| CCL4 | NM_002984.4 | NP_002975.1 | C-C motif chemokine |
| | | | ligand 4 |
| CCL5 | NM_001278736.2 | NP_001265665.1 | C-C motif chemokine |
| | | | ligand 5 |
| CCL6 | NM_009139.3 | NP_033165.1 | C-C motif chemokine |
| | | | ligand 6 |
| CCL7 | NM_006273.4 | NP_006264.2 | C-C motif chemokine |
| | | | ligand 7 |
| GAS6 | NM_000820.4 | NP_000811.1 | growth arrest specific 6 |
| LOX | NM_002317.7 | NP_002308.2 | lysyl oxidase |

Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers. (See U.S. Pat. Nos. 5,118,937; 5,045,694; 5,719,060; 6,225,047)

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of Clec7. Measurement of the level of a biomarker can be direct or indirect. For example, the abundance levels of Clec7 can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers useful in the present methods.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment.

Gene arrays are prepared by selecting probes which comprise a polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

Alternatively, levels of Translocator Protein (TSPO) can be used, e.g., as determined using TSPO imaging methods, e.g., positron emission tomography (PET) imaging of the brain of the subject. See, e.g., Werry et al., Int J Mol Sci. 2019 July; 20(13): 3161. As shown herein, TSPO correlates with the presence of MGnD.

In some embodiments, the presence and/or level of the inflammatory biomarker (e.g., Clec7/TSPO or another inflammatory biomarker listed herein) is comparable to the presence and/or level of the protein(s) in the disease reference, and the subject has one or more symptoms associated with neurodegeneration, then the subject is likely to respond to treatment and/or is selected for treatment using a method described herein. In some embodiments, the subject has no overt signs or symptoms of neurodegeneration but the presence and/or level of the inflammatory biomarker is comparable to the presence and/or level of the protein(s) in the disease reference, then the subject can be selected and/or treated using a method described herein. In some embodiments, such subjects are those who are at risk of developing a neurodegenerative disease, due to the presence of one or more risk factors such as age, family history, genetic predisposition, and presence of cardiovascular conditions such as heart disease, diabetes, stroke, high blood pressure and high cholesterol.

Suitable reference values can be determined using methods known in the art, e.g., using standard clinical trial methodology and statistical analysis. The reference values can have any relevant form. In some cases, the reference comprises a predetermined value for a meaningful level of the inflammatory biomarker, e.g., a control reference level that represents a normal level of the inflammatory biomarker, e.g., a level in an unaffected subject or a subject who is not at risk of developing a disease described herein and thus who would not benefit from a treatment described herein, and/or a disease reference that represents a level of the inflammatory biomarker associated with conditions associated with neurodegenerative disease and likelihood of response to treatment using a method described herein, e.g., a level in a subject having AD, ALS, or MS.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Alterations in levels of the inflammatory biomarker, e.g., Clec7 or TSPO, after administration of a treatment using a method described herein can be used to monitor treatment efficacy. Thus the present methods can include obtaining a baseline/pre-treatment level of the inflammatory biomarker in the subject, administering one or more doses of Xe to the subject, and obtaining a subsequent level the inflammatory biomarker. A decrease in the level of the inflammatory biomarker indicates that the treatment has been successful. An increase or no change in the level of the inflammatory biomarker can indicate that the treatment has not yet been successful, and can indicate that the dose amount and/or frequency of treatment should be increased. These methods can also be used to monitor a subject long term to determine whether treatment with Xe should be stopped (e.g., when levels of the inflammatory biomarker fall below a selected threshold) or resumed (e.g., when levels of the inflammatory biomarker increase, e.g., after a period during which they were below a selected threshold).

### EXAMPLES

The invention is further described in the following examples, which do not limit the scope of the invention described in the claims.

### Example 1. Reciprocal Induction of APOE and Suppression of TGFβ Signaling in Disease-Associated Microglia

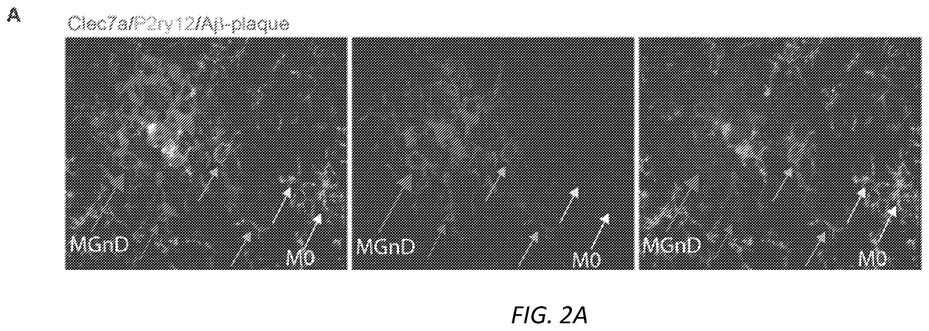

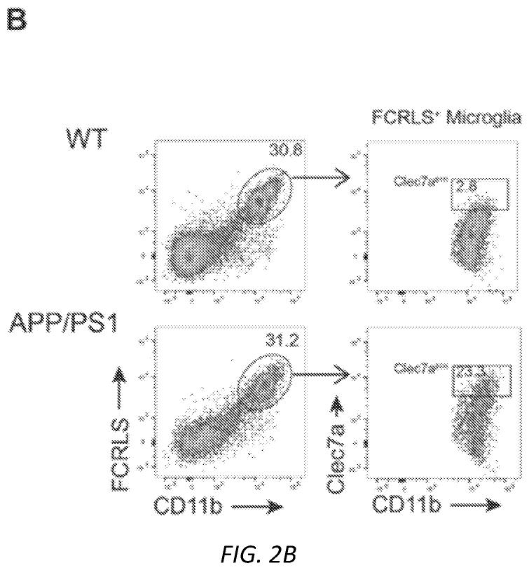

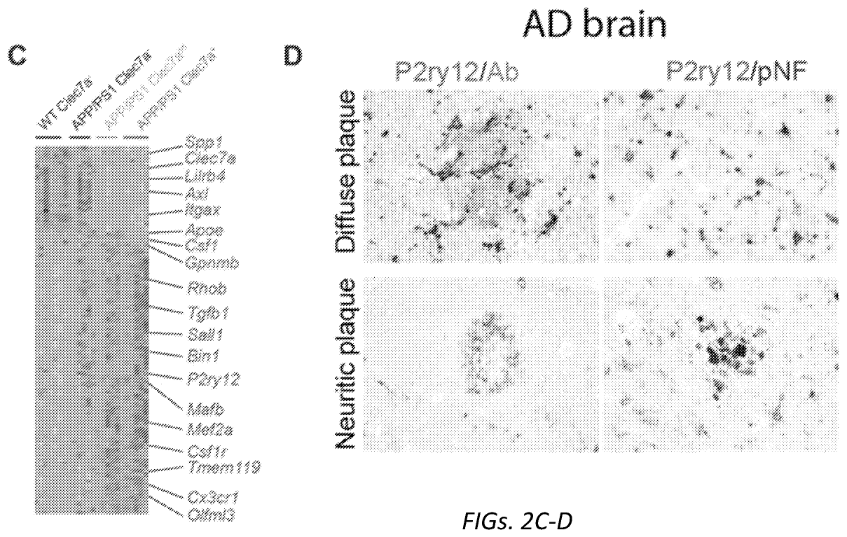

To investigate the molecular mechanisms that regulate microglial phenotype and function in disease, we analyzed FACS-sorted FCRLS⁺ microglia by RNAseq in different disease stages of mouse models of ALS (SOD1), AD (APP-PS1) and EAE. We identified a molecular signature that was commonly dysregulated in all three mouse models during both acute and chronic disease stages⁶. This common gene-cluster contains suppressed key microglia homeostatic genes including P2ry12, Tgfbr1, Gpr34, Jun, Olfml3, Csflr, Hexb, Mertk, Rhob, Cx3Cr1 and Tgfb1, and upregulated inflammatory genes including Spp1, Itgax, Axl, Lilrb4, Clec7a, Tlr2, Ccl2, Csfl and Bhlhe40 (FIG. 2C). Apoe was the most upregulated gene. Moreover, the expression of Apoe correlated with disease progression in EAE, SOD1 and APP-PS1 mice as compared to WT littermates and during aging. Ingenuity pathway analysis (IPA) identified reciprocal induction of APOE and suppression of TGFβ signaling as the major regulators in MGnD microglia.

### Example 2. Loss of Homeostatic Microglia in APP/PS1 Mice

We investigated whether microglial phenotype switch from M0 to MGnD is associated with neuritic dystrophy, which is a hallmark of AD pathology. We identified three microglia subsets: 1) Clec7a⁻P2ry12⁺ (not associated with Aβ plaques); 2) Clec7a^{lo}P2ry12^{lo} (in close proximity to Aβ plaques); 3) Clec7a⁺P2ry12⁻ (associated with Aβ plaques) (FIGS. 2A-B)⁶. Interestingly, APOE expression correlates with increased expression of Clec7a^{+ microglia associated with Aβ-neuritic plaques. RNA-seq analysis of plaque-associated Clec}7a⁺/FCRLS⁺ microglia and non-plaque Clec7a⁻/FCRLS⁺ microglia from APP-PS1 mice showed induction of MGnD and suppression of M0 molecular signatures in Clec7a⁺ microglia (FIG. 2C). Finally, we confirmed suppression of P2ry12 in microglia-associated neuritic plaques in AD human brain (FIG. 2D). Thus, during both aging and in AD, impaired microglial sensome functions^{26,27} can be linked to microglial impaired homeostatic molecular signature and functions. Importantly, induction of MGnD-like phenotype in microglia isolated from human AD brains is similar to microglia isolated from a mouse model of AD⁶. Therefore, 1) APP/PS1 mice is a good model to investigate microglial phenotype which is similar to microglia from human AD brain and 2) immunomodulatory approaches to restore M0-microglial phenotype and function provide a potential therapy to treat AD.

### Example 3. Xe-Gas Treatment Protects Neurons from Apoptosis

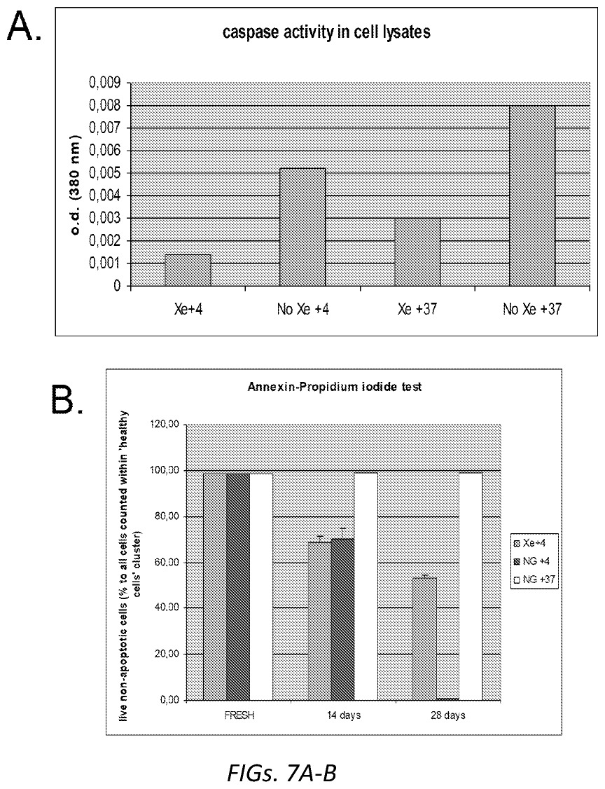

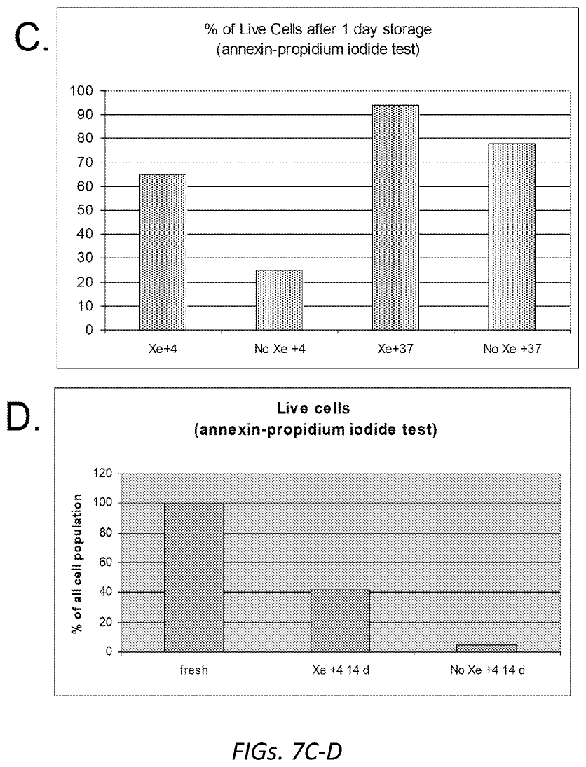

It is known that Xe treatment protects neurons from apoptosis by reducing intracellular calcium, protecting mitochondrial membranes, and attenuating cell damage caused by reactive oxygen species (ROS)^{28,29}. We found profound anti-apoptotic effect of Xe-gas on the variety of biological cells including platelets and red blood cells, Lymphocyte-like Jurkat cells, H9C2 and U937 cells (human macrophage cell lines); see FIGS. 7A-D. Other groups also reported direct antiapoptotic effect of Xe on neurons in AD cell models, specifically cortical neurons and basal forebrain cholinergic neurons submitted to mild excitotoxic stress by continued exposure to L-trans-pyrrolidine-2,4-dicarboxylic acid (PDC), a synthetic analog of L-glutamate¹⁴.

### Example 4. Xenon Treatment Suppresses MGnD-Neurodegenerative Microglia and Restores M0-Homeostatic Microglia in APP/PS1 Mice

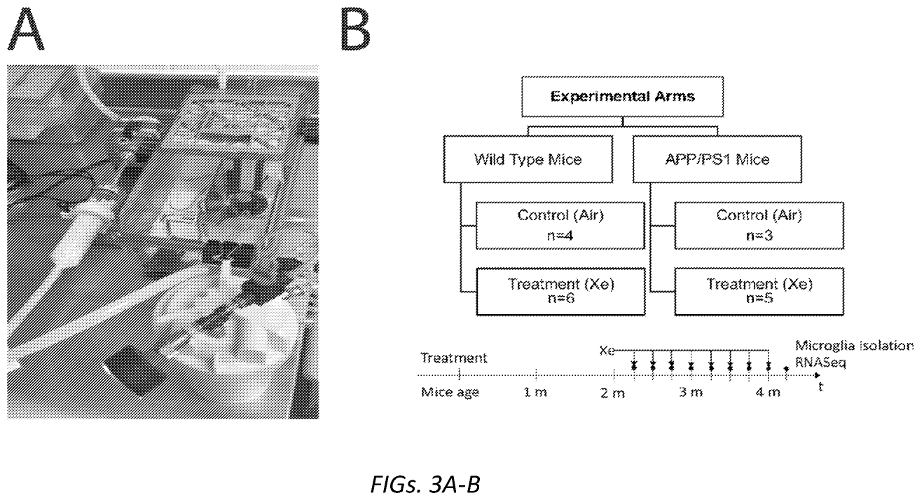

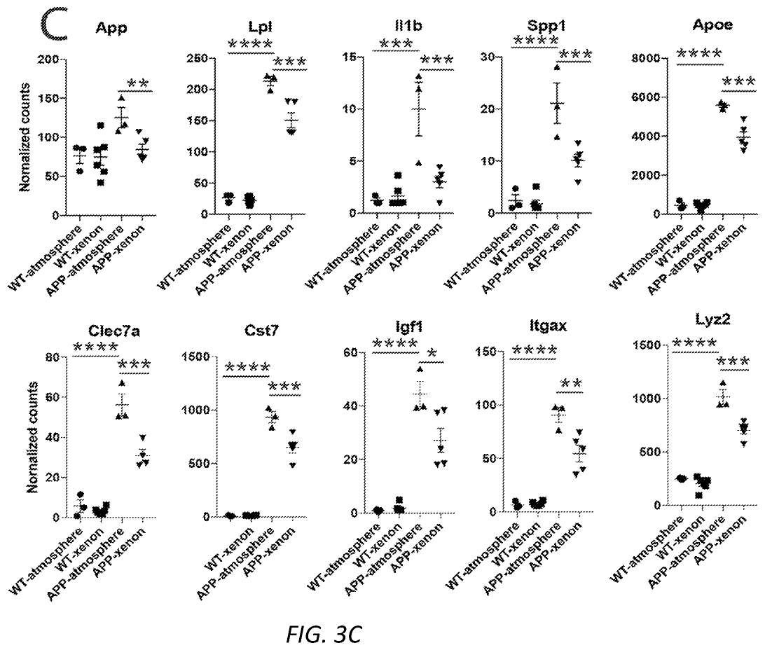

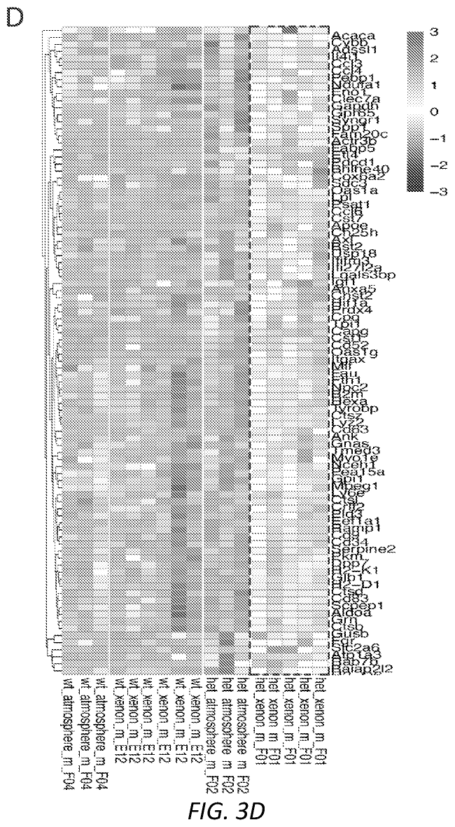

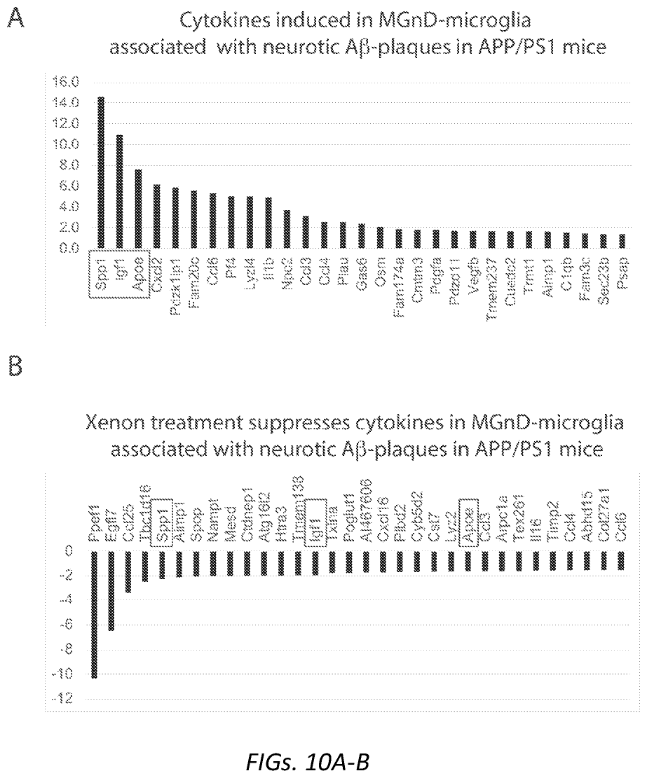

Based on recent findings that danger signals (dead neurons, neuritic axons and Ab peptides) alter functional phenotype of microglia from the homeostatic (M0) to neurodegenerative (MGnD) phenotype in AD, we hypothesized that Xe-gas treatment has a protective immunomodulatory role to restore homeostatic ‘sensome’ microglial functions in neurodegenerative diseases. To address whether Xe-treatment will restore functional microglia and suppress AD-like pathology in APP-PS1³⁰ mice, we started treatment with Xe at 2 months of age (at the onset of amyloid deposition—early stage) for 8 weeks (once a week for 40 minutes). Of note, these mice start to develop plaques at 6 weeks of age with saturation around 8 months of age. To treat mice with xenon gas we constructed a special chamber which can control supply of Xe and atmospheric gas (FIG. 3A). The closed-circuit system pumps recirculated gas through soda-lime to absorb CO2. CO2 and O2 concentrations were continuously monitored. The gas mixture was premixed in the outside container with excessive pressure. That lead to approximately 70% Xe and 20% 02 in the chamber. When 02 concentration reduced, additional gas mixture was supplied from the mixing container. The system was optimized to treat simultaneously up to 6 mice. Two types of mice were used: APP/PS1 mice and Wild Type mice as a control. The groups contained both males and females who were housed and treated separately. In each group we subjected portion of the mice to Xe treatment, and another portion was used as control by treatment with atmospheric air (FIG. 3B). We isolated FCRLS⁺ microglia by FACS and performed RNAseq analysis. DESeq2 analysis identified 1,206 genes significantly affected among the experimental groups. FIG. 3C shows selected cluster of the core MGnD significantly affected genes. Xe treatment suppressed inflammatory genes including App, Lpl, IIlb, Spp1 and Apoe (FIGS. 3D and 10A-B). Moreover, Bhlhe40, which is essential for pathogenicity in neuroinflammation^{9,10}, was significantly reduced in microglia from APP/PS1 treated with Xe.

### Example 5. Xenon Treatment Suppresses MGnD-Neurodegenerative Microglia and Restores M0-Homeostatic Microglia in Acute Neurodegeneration Mouse Model

We developed a new acute neurodegeneration mouse model to induce neurodegenerative MGnD-microglia³⁶. In brief, primary neurons (d7-d10) were typically cultured one week after initiation of culture. Neurons were removed from the surface of the plate by multiple washed with PBS. The neurons were then incubated under UV light (302 nm) at an intensity of 6315 W for 15 minutes to induce apoptosis. After this step the neurons were kept on ice. The cells were collected, spun down via centrifugation, and resuspended in 1 ml PBS. Next they were stained with the labeling dye (Alexa405 NHS Ester, Invitrogen, A3000) for 15 min at 37° C., protected from light. Neurons were then washed, spun and resuspended. Number of apoptotic cells was determined using a trypan blue stain and a cellometer. Neurons were resuspended at a density of 50,000 cells per μl.

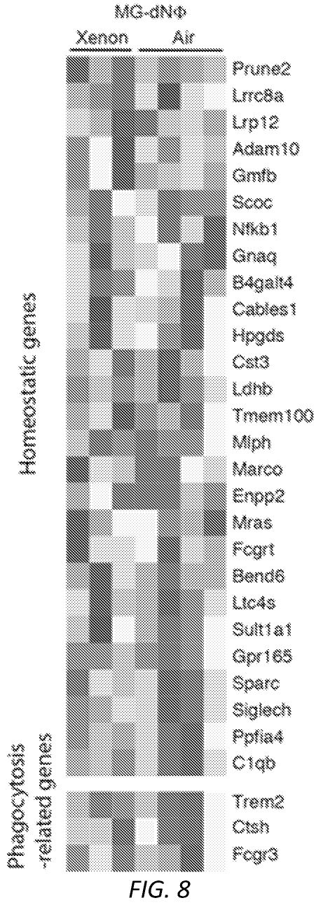

These apoptotic dead neurons (dN) injected into the cortex and hippocampus of naïve mice induced the recruitment of P2ry12⁺ microglia towards the site of injection. P2ry12⁺ microglia changed morphology from an M0-homeostatic non-phagocytic (MG-nΦ) phenotype to an amoeboid-phagocytic (MG-dNΦ) phenotype at the vicinity of the injection site. Induction of MGnD microglia was not detected in PBS-injected control brains. Utilizing this model, we found that mice treated with Xe-gas for one hour immediately after dead neurons injection induces expression of homeostatic genes such as Siglech, Cst3, Fcgrt and Clqb. Importantly, Xe treatment significantly induced Trem2 expression in microglia (see FIG. 8). Reducing Trem2 expression has been shown previously, including by our laboratory and others, to have beneficial effect on microglia recruitment towards Aβ-plaques⁶¹⁻⁶³, associated with restriction of AD⁶⁴. Moreover, TREM2 has been recently identified as a receptor for Aβ⁶⁵. Therefore, these data demonstrated that Xe-gas treatment modulates microglial phenotype and pushes the balance towards repair. This provides evidence that Xe-gas treatment has a protective immunomodulatory role to induce microglia protective functions to treat AD.

### Example 6. Xenon Treatment Reduced Clec7a⁺ MGnD-Neurodegenerative Microglia Subset and Ab Load in APP/PS1 Mice

APP-PS1 and WT mice were sacrificed and perfused with Hanks' Balanced Salt solution (HBSS). The mouse brains were separated, and the right hemispheres fixed in 4% PFA. After 24 hours the hemispheres were then transferred to a 30% sucrose solution for 48 hours. Brains were later frozen in Tissue-TeK O.C.T. and stored at −80 until ready to be cut. 30 mm sagittal brain sections were cut with a cryostat and kept free floating in cryoprotectant solution. On the first day of staining, free floating brain sections were washed twice with phosphate buffered saline (PBS) and then incubated for one hour in blocking buffer (PBS, 0.1% Triton, 1% Bovine Serum Albumin, 10% Normal Horse Serum). Afterwards, the sections were incubated with primary antibody [Mouse-anti-Abeta Ab (1:1000, Biolegend, 6E10) and Rat-anti-Clec7a (1:300, Invivogen, mabg-mdect)] in pre-blocking buffer (PBS, 0.1% Triton, 1% Bovine Serum Albumin, 5% Normal Horse Serum) on a shaker overnight at 4° C. The following day, the sections were washed three times with PBS. Secondary antibodies (Donkey-anti-mouse-Cy3 (1:800, JIR, 715-165-150) and Goat-anti-Rat-647 (1:800, Invitrogen, A-21247)) in pre-blocking buffer were added to the sections for 1 hour at room temperature. After washing the sections 3 times with PBS, they were moved onto glass slides and covered with a glass coverslip and DAPI. Then images were taken by Leica DMi8 microscope. 4-6 images of prefrontal cortex with 10× magnification were taken per brain section (3 brain sections were stained per mouse, n=3-5).

For quantification of MGnD and Ab load, acquired images were imported to Fiji software (ImageJ). Then the data channels were separated. Gaussian filtering was used to remove noise. Next, the MGnD and plaques were shown using automatic thresholding methods in Fiji (with “Triangle” thresholding setting for MGnD and “Otsu” for Ab load 6E10). Finally, the percentage of the MGnD and Ab plaques over the cortical area was calculated and averaged, indicating the distribution of MGnD and Ab plaques in the prefrontal cortex.

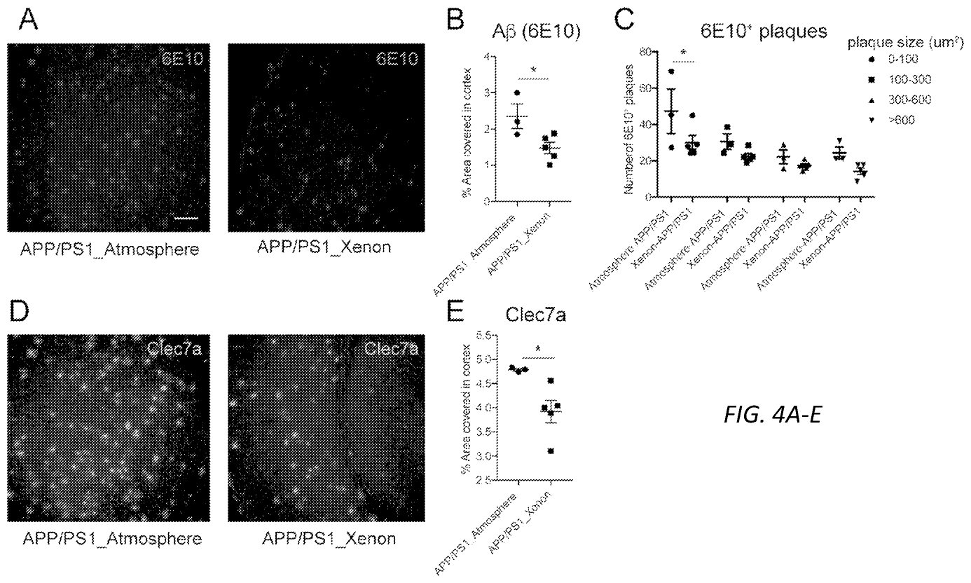

As shown in FIGS. 4A-B, there was a significant reduction of % cortical area of 6E10+ Abeta plaques in Xenon-treated group, compared to the control group. We further quantified the number of plaques of different sizes. It showed there was a reduction in all groups of different plaque sizes and the reduction reached significance in the group of plaque size as 0-100 um2 (FIG. 4C). As shown in FIG. 4D, E, Xenon treatment significantly decreased % positive area stained by Clec7a, compared to the control group. These results demonstrated Xe-gas treatment decreased MGnD microglia subset and Abeta load in APP/PS1 mice. This provides evidence that Xe-gas treatment can be used as a potential therapeutic to treat AD.

### Example 7. Xenon Treatment Decreased Apoe Phagocytosed by CD68+ Phagosomes in APP/PS1 Mice

We identified two microglial subsets in 24-month-old APP-PS1 mice by FACS sorting: Clec7a⁺FCRLS⁺ microglia (associated with neuritic Ab plaques) and Clec7a⁻FCRLS⁺ microglia (not associated with Ab plaques). The mice were perfused with cold HBSS. The whole brain was removed from the mouse and the left hemisphere was homogenized to form a single cell suspension, then resuspended and centrifuged in a 37%/70% Percoll Plus (GE Healthcare, 17-5445-02) gradient at 800G, 23° C., for 25 min with an acceleration of 3 and a deceleration of 1. Mononuclear cells were taken from the interface layer. The cells were stained with anti-Clec7a (1:10, Invivogen, mabg-mdect) followed by secondary detection with goat anti-rat IgG conjugated to FITC (1:300, Biolegend, clone poly4054), anti-Fcrls-APC (1:800), anti-CD11b-PeCy7 (1:300, eBioscience, 50-154-54), and anti-mouse Ly-6C-PerCP/Cy5.5 (1:300, Biolegend, 123012). After staining, cells were sorted using BD FACSAria™ II (BD Bioscience).

We compared the molecular signature of Clec7a^{+ vs Clec}7a⁻ vs WT microglia by High-coverage Smartseq2 RNA sequencing. The sentence could be changed as “Clec7a+ microglia represent the MGnD microglia identified in SOD1, EAE, and APP-PS1 models and during aging⁵. The significantly upregulated genes include Spp1, Itgax, Axl, Lilrb4, Apoe, Clec7a, and Tspo.

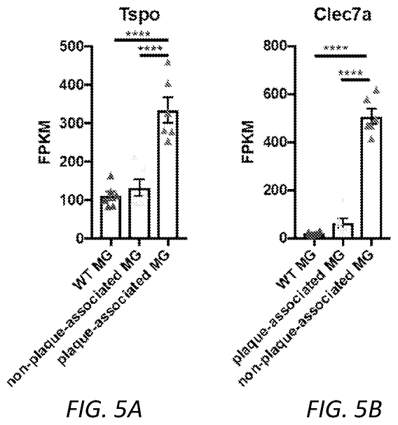

As shown in FIGS. 5A-B, MGnD associated with Ab plaques specifically express Clec7a, which is correlated with induction of Translocator Protein (TSPO). Thus, TSPO PET-based imaging can be used to stratify AD patients for Xe treatment.

These results demonstrate that TSPO imaging can be used to monitor the effect of Xe on restoration of homeostatic microglia in AD patients and to optimize treatment protocol.

### Example 8. Xenon Treatment Decreased Apoe Phagocytosed by CD68+ Phagosomes in APP/PS1 Mice

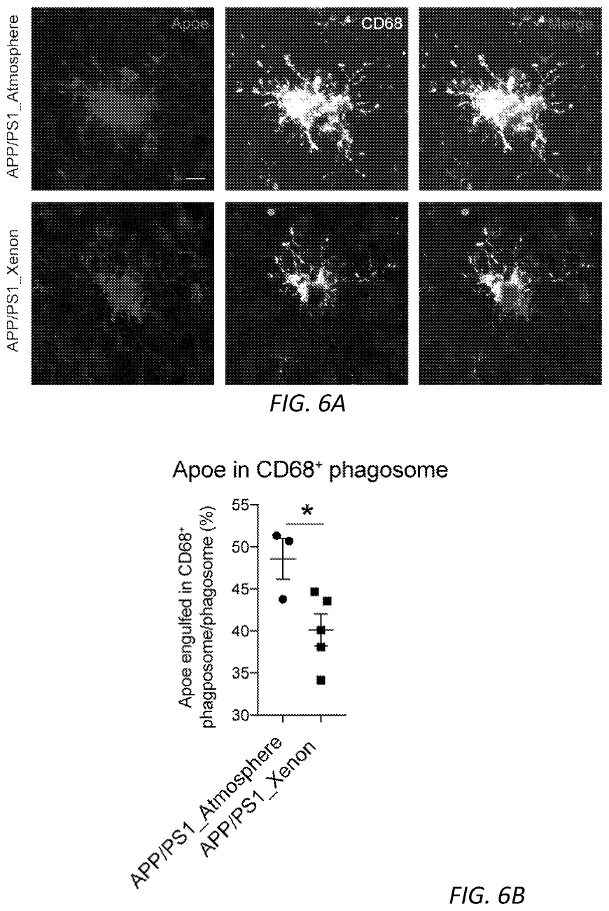

APP-PS1 and WT mice were exposed to Xenon vs. normal air in vivo for 8 weeks. After that, the mice were sacrificed and perfused with Hanks' Balanced Salt solution (HBSS). The mouse brain was separated, and the right hemisphere was fixed in 4% PFA. After 24 hours the hemisphere was then transferred to a 30% sucrose solution for 48 hours. Brains were later frozen in Tissue-TeK O.C.T. and stored at −80 until ready to be cut. 30 mm sagittal brain sections were cut with a cryostat and kept free floating in cryoprotectant solution. On the first day of staining, free floating brain sections were washed twice with phosphate buffered saline (PBS) and then incubated for one hour in blocking buffer (PBS, 0.1% Triton, 1% Bovine Serum Albumin, 10% Normal Horse Serum). Afterwards, the sections were incubated with primary antibody [Mouse-anti-Apoe (1:1000) and Rat-anti-CD68 (1:200)] in pre-blocking buffer (PBS, 0.1% Triton, 1% Bovine Serum Albumin, 5% Normal Horse Serum) on a shaker overnight at 4° C. The following day, the sections were washed three times with PBS. Secondary antibodies [Donkey-anti-mouse-Cy3 (1:800, JIR, 715-165-150) and Goat-anti-Rat-647 (1:800, Invitrogen, A-21247)] in pre-blocking buffer were added to the sections for 1 hour at room temperature. After washing the sections 3 times with PBS, they were moved onto glass slides and covered with a glass coverslip and DAPI. The images were taken by Zeiss LSM 710 confocal microscope. 5-9 plaques per sample were selected and imaged using Z-stack. For quantification of the percentage of Apoe engulfed in CD68⁺ phagosomes, acquired images were imported to Fiji software (ImageJ). Then the data channels were separated. Gaussian filtering was used to remove noise. Next, the Apoe and CD68 were shown using automatic thresholding method “Otsu” in Fiji. The overlap of the staining/CD68+ staining was quantified in both groups. Data are presented as mean±SEM. Student's t-test was used for statistics. * p<0.05. The results shown in FIGS. 6A, demonstrated APOE in the plaque regions were engulfed by CD68⁺ phagosomes in both Xe-gas treated and control groups. The results shown in FIGS. 6B, demonstrated there was a significant reduction of APOE phagocytosed by CD68⁺ cells.

The results indicated that the Xenon treatment greatly modulated microglial phenotype and phagocytic functions. Xenon treatment reduced APOE in the plaque region to treat AD.

### Example 9. Xenon Treatment in EAE Model

In order to evaluate the effects of Xenon as a possible candidate for Multiple Sclerosis (MS) treatment, we development a new protocol of exposition. We used the experimental autoimmune encephalomyelitis (EAE) model that mimics part of the mechanisms presents in MS. The induction of this model is due to the combination of the oligodendrocyte myelin (MOG) diluted in complete Freund's adjuvant (CFA) to break immunological tolerance and initiate the development of autoimmunity in these animals. In association we give two 200 ng doses of Bordetella Pertussis toxin that helps in increasing the permeability of the blood-brain barrier allowing the entry of immune cells to the spinal cord.

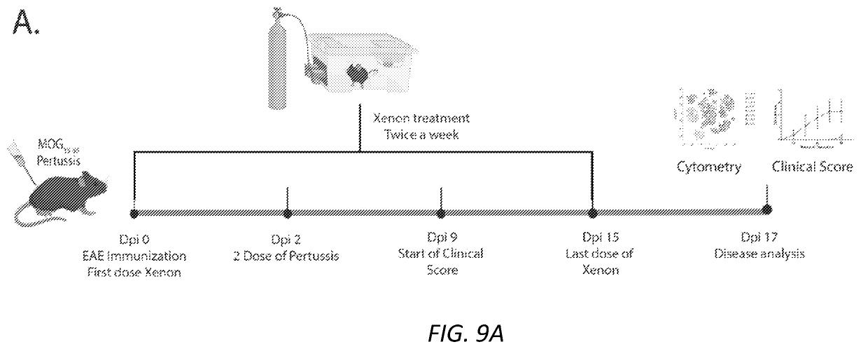

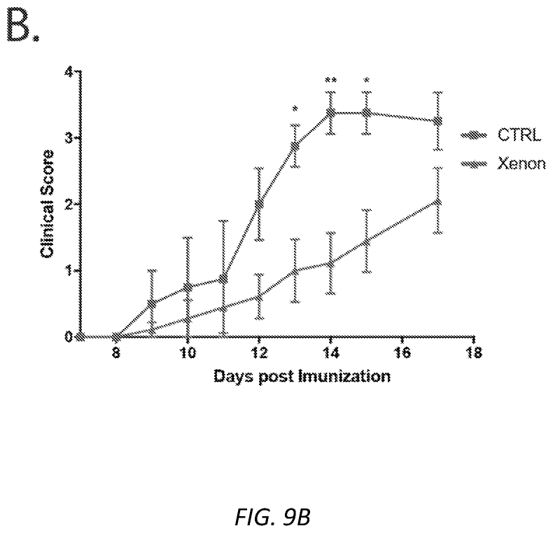

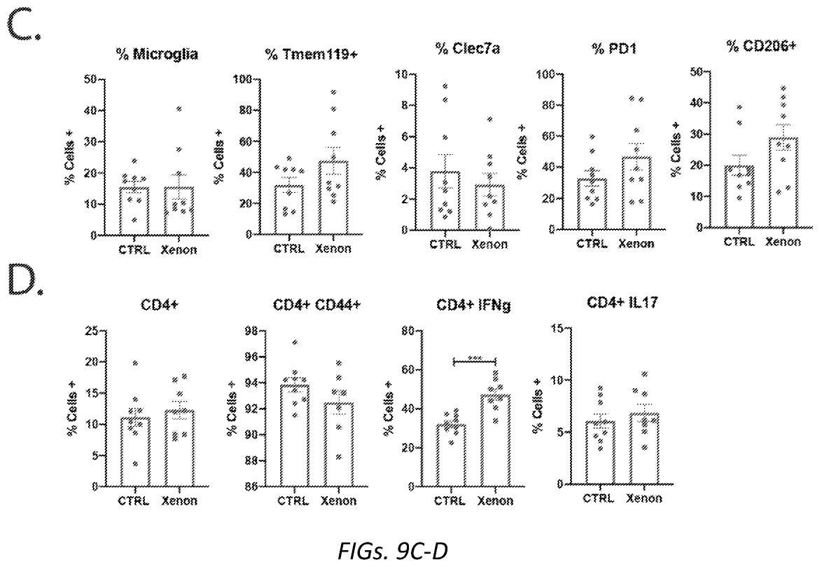

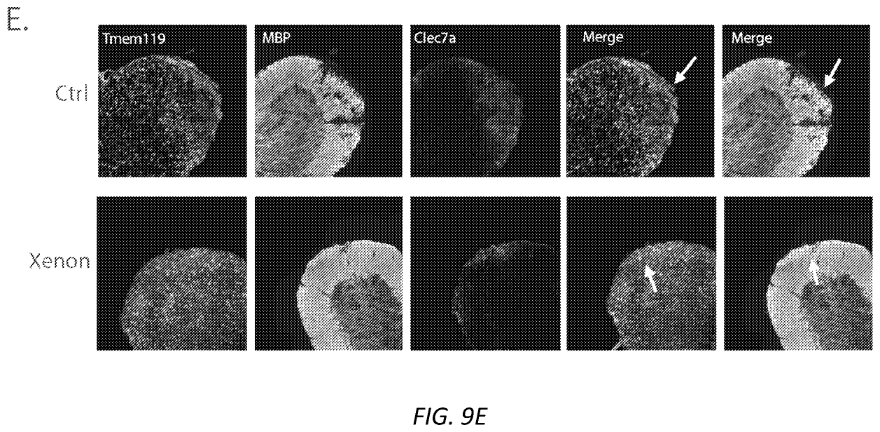

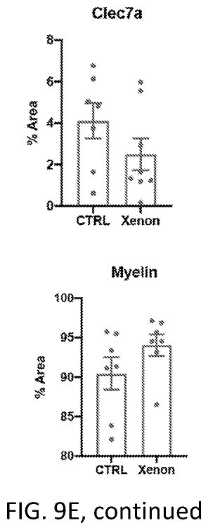

The treatment with xenon was divided into 5 exposures of 40 minutes divided twice a week in the concentration of 3 PSI of xenon with 1 PSI of oxygen with the oxygen and CO2 concentration constantly measured. This treatment took place from day 0 to day 15 after immunization, with the animals' euthanasia on day 17 (See FIG. 9A). The clinical evaluation started on day 8 with the clinical signs being classified into: 0 no disease, 1—limp tail, 2—weak/partially paralyzed hind legs, 3—completely paralyzed hind legs, 4—complete hind and partial front leg paralysis, 5—complete paralysis/death.

At the end of the experiment, we observed that treatment with xenon was able to delay the clinical signs (FIG. 9B). The microglial cells of the treated animals tended to have a higher frequency of positive cells for Tmem119, PD1 and CD206 and a lower frequency of positive cells for Clec7a indicating a more suppressive response (FIG. 9C). In addition, T lymphocytes, despite presenting a higher frequency of IFNg, had a tendency to lower expression of CD44 (activation marker) (FIG. 9D), a fundamental cytokine for their pathogenic response in this model. In the immunohistochemistry analyses we observed a tendency of less demyelination associated with a decrease in the frequency of clec7a+ cells in the spinal cord of treated animals (FIG. 9E). These results indicate a lower activation of immune cells accompanied by a lesser sequela in the animals.

### REFERENCES

1 Cruchaga, C. et al. Cerebrospinal fluid APOE levels: an endophenotype for genetic studies for Alzheimer's disease. Hum Mol Genet 21, 4558-4571, doi:10.1093/hmg/dds296 (2012).
2 Hickman, S. E. et al. The microglial sensome revealed by direct RNA sequencing. Nat Neurosci 16, 1896-1905, doi:10.1038/nn.3554 (2013).
3 Butovsky, O. et al. Identification of a unique TGF-beta-dependent molecular and functional signature in microglia. Nat Neurosci 17, 131-143, doi: 10.1038/nn.3599 (2014).
4 Gautier, E. L. et al. Gene-expression profiles and transcriptional regulatory pathways that underlie the identity and diversity of mouse tissue macrophages. Nat Immunol 13, 1118-1128, doi:10.1038/ni.2419 (2012).
5 Chiu, I. M. et al. A neurodegeneration-specific gene-expression signature of acutely isolated microglia from an amyotrophic lateral sclerosis mouse model. Cell Rep 4, 385-401, doi:10.1016/j.celrep.2013.06.018 (2013).
6 Krasemann, S. et al. The TREM2-APOE Pathway Drives the Transcriptional Phenotype of Dysfunctional Microglia in Neurodegenerative Diseases. Immunity 47, 566-581 e569, doi:10.1016/j.immuni.2017.08.008 (2017).
7 Gosselin, D. et al. Environment drives selection and function of enhancers controlling tissue-specific macrophage identities. Cell 159, 1327-1340, doi:10.1016/j.cell.2014.11.023 (2014).
8 Matcovitch-Natan, O. et al. Microglia development follows a stepwise program to regulate brain homeostasis. Science 353, aad8670, doi:10.1126/science.aad8670 (2016).
9 Lin, C. C. et al. Bhlhe40 controls cytokine production by T cells and is essential for pathogenicity in autoimmune neuroinflammation. Nat Commun 5, 3551, doi:10.1038/ncomms4551 (2014).
10 Sun, H., Lu, B., Li, R. Q., Flavell, R. A. & Taneja, R. Defective T cell activation and autoimmune disorder in Stra13-deficient mice. Nat Immunol 2, 1040-1047, doi:10.1038/ni721 (2001).
11 Shi, Y. et al. ApoE4 markedly exacerbates tau-mediated neurodegeneration in a mouse model of tauopathy. Nature 549, 523-527, doi:10.1038/nature24016 (2017).
12 David, H. N. et al. Ex vivo and in vivo neuroprotection induced by argon when given after an excitotoxic or ischemic insult. PLoS One 7, e30934, doi:10.1371/journal.pone.0030934 (2012).
13 Lavaur, J. et al. The noble gas xenon provides protection and trophic stimulation to midbrain dopamine neurons. J Neurochem 142, 14-28, doi:10.1111/jnc.14041 (2017).
14 Lavaur, J. et al. Xenon-mediated neuroprotection in response to sustained, low-level excitotoxic stress. Cell Death Discov 2, 16018, doi:10.1038/cddiscovery.2016.18 (2016).
15 Ulbrich, F. et al. Argon Mediates Anti-Apoptotic Signaling and Neuroprotection via Inhibition of Toll-Like Receptor 2 and 4. PLoS One 10, e0143887, doi:10.1371/journal.pone.0143887 (2015).
16 Begcevic, I., Brinc, D., Drabovich, A. P., Batruch, I. & Diamandis, E. P. Identification of brain-enriched proteins in the cerebrospinal fluid proteome by LC-MS/MS profiling and mining of the Human Protein Atlas. Clin Proteomics 13, 11, doi:10.1186/s12014-016-9111-3 (2016).
17 Luo, Y. et al. Elevated Levels of NLRP3 in Cerebrospinal Fluid of Patients With Autoimmune GFAP Astrocytopathy. Front Neurol 10, 1019, doi:10.3389/fneur.2019.01019 (2019).
18 Meyerhoff, N., Rohn, K., Carlson, R. & Tipold, A. Measurement of Neutrophil Gelatinase-Associated Lipocalin Concentration in Canine Cerebrospinal Fluid and Serum and Its Involvement in Neuroinflammation. Front Vet Sci 6, 315, doi:10.3389/fvets.2019.00315 (2019).
19 Lepennetier, G. et al. Cytokine and immune cell profiling in the cerebrospinal fluid of patients with neuro-inflammatory diseases. J Neuroinflammation 16, 219, doi:10.1186/s12974-019-1601-6 (2019).
20 Wang, C. et al. CXCL13, CXCL10 and CXCL8 as Potential Biomarkers for the Diagnosis of Neurosyphilis Patients. Sci Rep 6, 33569, doi:10.1038/srep33569 (2016).
21 Hirai, T. et al. Increased plasma lipoprotein lipase activity in males with autism spectrum disorder. Research in Autism Spectrum Disorders 77, doi:10.1016/j.rasd.2020.101630 (2020).
22 Fainardi, E. et al. Cerebrospinal fluid and serum levels and intrathecal production of active matrix metalloproteinase-9 (MMP-9) as markers of disease activity in patients with multiple sclerosis. Mult Scler. 2006 June; 12(3):294-301, doi: 10.1191/135248506ms1274oa.
23 Mikhael, N. L., Gendi, M. A. S. H., Hassab, H. & Megahed, E. A. Evaluation of multiplexed biomarkers in assessment of CSF infiltration in pediatric acute lymphoblastic leukemia. Int J Hematol Oncol. (2019).
24 Taipa, R. et al. Proinflammatory and anti-inflammatory cytokines in the CSF of patients with Alzheimer's disease and their correlation with cognitive decline. Neurobiol Aging 76, 125-132, doi:10.1016/j.neurobiolaging.2018.12.019 (2019).
25 Sainaghi, P. P. et al. Elevation of Gas6 protein concentration in cerebrospinal fluid of patients with chronic inflammatory demyelinating polyneuropathy (CIDP). J Neurol Sci 269, 138-142, doi:10.1016/j.jns.2008.01.005 (2008).
26 Streit, W. J. Microglial senescence: does the brain's immune system have an expiration date? Trends in neurosciences 29, 506-510 (2006).
27 Streit, W. J., Braak, H., Xue, Q. S. & Bechmann, I. Dystrophic (senescent) rather than activated microglial cells are associated with tau pathology and likely precede neurodegeneration in Alzheimer's disease. Acta neuropathologica 118, 475-485, doi:10.1007/s00401-009-0556-6 (2009).
28 Liu, W., Khatibi, N., Sridharan, A. & Zhang, J. H. Application of medical gases in the field of neurobiology. Med Gas Res 1, 13, doi: 10.1186/2045-9912-1-13 (2011).
29 Franks, N. P., Dickinson, R., de Sousa, S. L., Hall, A. C. & Lieb, W. R. How does xenon produce anaesthesia? Nature 396, 324, doi:10.1038/24525 (1998).
30 Radde, R. et al. Abeta42-driven cerebral amyloidosis in transgenic mice reveals early and robust pathology. EMBO Rep 7, 940-946, doi:10.1038/sj.embor.7400784 (2006).
31 Mazaheri, F. et al. TREM2 deficiency impairs chemotaxis and microglial responses to neuronal injury. EMBO Rep 18, 1186-1198, doi:10.15252/embr.201743922 (2017).
32 Ulland, T. K. et al. TREM2 Maintains Microglial Metabolic Fitness in Alzheimer's Disease. Cell 170, 649-663 e613, doi:10.1016/j.cell.2017.07.023 (2017).
33 Wang, Y. et al. TREM2 lipid sensing sustains the microglial response in an Alzheimer's disease model. Cell 160, 1061-1071, doi:10.1016/j.cell.2015.01.049 (2015).
34 Keren-Shaul, H. et al. A Unique Microglia Type Associated with Restricting Development of Alzheimer's Disease. Cell 169, 1276-1290 e1217, doi:10.1016/j.cell.2017.05.018 (2017).
35 Zhao, Y. et al. TREM2 Is a Receptor for beta-Amyloid that Mediates Microglial Function. Neuron 97, 1023-1031 e1027, doi:10.1016/j.neuron.2018.01.031 (2018).
36 Buttgereit, A. et al. Sall1 is a transcriptional regulator defining microglia identity and function. Nat Immunol 17, 1397-1406, doi:10.1038/ni.3585 (2016).
37 Veldeman, M. et al. Xenon Reduces Neuronal Hippocampal Damage and Alters the Pattern of Microglial Activation after Experimental Subarachnoid Hemorrhage: A Randomized Controlled Animal Trial. Front Neurol 8, 511, doi:10.3389/fneur.2017.00511 (2017).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject, the method comprising:
optionally identifying a subject who has a level of microglial cells that express C-type lectin domain family 7 member A (Clec7a) above a reference level; and
administering a therapeutically effective amount of xenon to the subject.

2. The method of claim 1, wherein the subject has, or is at risk of developing, a neurodegenerative disease.

3. The method of claim 2, wherein the neurodegenerative disease is Alzheimer's disease, Multiple Sclerosis (MS), or Amyotrophic Lateral Sclerosis (ALS).

4. The method of claim 1, wherein identifying a subject who has levels of Clec7a+ microglial cells above a reference level comprises:
measuring expression of translocator protein 18 kDa (TSPO) in a tissue of the subject, preferably in the brain of the subject, to determine a level of Clec7a+ microglial cells in the tissue; and
comparing the level of TSPO expression in the tissue to a reference level; and
identifying a subject who has a level of TSPO expression above the level as having a level of Clec7a+ microglial cells above the reference level.

5. The method of claim 4, further comprising determining a subsequent level of Clec7a+ microglial cells after administration of the xenon, and administering a further dose of xenon if the subsequent level of Clec7a+ microglial cells is above a reference level.

6. The method of claim 1, wherein identifying a subject who has levels of Clec7a+ microglial cells above a reference level comprises:
measuring levels of one or more inflammatory biomarkers selected from apolipoprotein E (APOE); secreted phosphoprotein 1 (SPP1); insulin like growth factor 1 (IGF1); NLR family pyrin domain containing 3 (NLRP3); cystatin C (CST3); cystatin D (CST5); cystatin F (CST7); lipocalin 2 (LCN2); C-X-C motif chemokine ligand 1 (CXCL1); C-X-C motif chemokine ligand 2 (CXCL2); C-X-C motif chemokine ligand 3 (CXCL3); C-X-C motif chemokine ligand 10 (CXCL10); colony stimulating factor 1 (CSF1); colony stimulating factor 3 (CSF3); lipoprotein lipase (LPL); integrin subunit alpha X (ITGAX); amyloid beta precursor protein (APP); lysozyme 2 (LYZ2); serpin family B member 2 (SERPINB2); matrix metallopeptidase 3 (MMP3); matrix metallopeptidase 9 (MMP9); matrix metallopeptidase 10 (MMP10); matrix metallopeptidase 13 (MMP13); cholesterol 25-hydroxylase (CH25H); interleukin 1 alpha (IL1A); interleukin 1 beta (IL1B); interleukin 12B (IL12B); interleukin 6 (IL6); tumor necrosis factor (TNF); endothelin 1 (EDN1); CD14; CD44; CD300 molecule like family member d (CD300LD); C-C motif chemokine ligand 2 (CCL2); C-C motif chemokine ligand 3 (CCL3); C-C motif chemokine ligand 4 (CCL4); C-C motif chemokine ligand 5 (CCL5); C-C motif chemokine ligand 6 (CCL6); C-C motif chemokine ligand 7 (CCL7); growth arrest specific 6 (GAS6); lysyl oxidase (LOX) in a sample from the subject, preferably a sample comprising blood from the subject to determine;
comparing the level of the inflammatory biomarker in the sample to a corresponding reference level; and
identifying a subject who has a level of APOE, SPP1, IGF1, NLRP3, CST3, CST5, CST7, LCN2, CXCL1, CXCL2, CXCL3, CXCL10, CSF1, CSF3, LPL, ITGAX, APP, LYZ2, SERPINB2, MMP3, MMP9, MMP10, MMP13, CH25H, IL1A, IL1B, IL12B, IL6, TNF, EDN1, CD14, CD44, CD300LD, CCL2, CCL3, CCL4, CCL5, CCL6, CCL7, or LOX that is above the reference level, or a level of GAS6 that is below the reference level as having a level of Clec7a+ microglial cells above the reference level.

7. The method of claim 1, wherein the xenon is administered to the subject in a gas for inhalation.

8. The method of claim 7, wherein the gas comprises at least 25%, 30%, 35%, 40%, 45%, 50%, 55%, 60%, 65%, up to 70% xenon, and at least 20%, 21%, 25%, 30%, or 40% oxygen.

9. The method of claim 1, wherein the xenon is administered for at least 30 minutes, 45 minutes, one hour, or two hours.

10. The method of claim 1, wherein the xenon is administered daily, once a week, twice a week, every other week, once a month, or once every two months.

11. The method of claim 9, wherein the xenon is administered once, twice, three times, or four times a week or more.

12. The method of claim 10, wherein the administration is repeated for at least two, three, four, five, 6, 7, 8, 9, 10, 11, or 12 weeks, six months, a year, or more.

13. The method of claim 10, wherein the administration is repeated every other week for at least eight weeks.

14. The method of claim 1, wherein the treatment reduces levels of Clec7a+ microglial cells in the subject.

15. The method of claim 1, wherein the treatment reduces inflammation in the subject.

16. The method of claim 15, wherein the inflammation is neuroinflammation.

17.-32. (canceled)
